(19) United States
(12) Patent Application Publication
(10) Pub. No.: US 2019/0215368 A1
(43) Pub. Date: Jul. 11, 2019

(54) Internet of Things (“IoT”)-Enabled Toothbrush Device to Monitor Human Vital Signs

(71) Applicants: Jacqueline Thanh-Thao Do, Cupertino, CA (US); Tiffany Quynh-Nhi Do, Cupertino, CA (US)

(72) Inventors: Jacqueline Thanh-Thao Do, Cupertino, CA (US); Tiffany Quynh-Nhi Do, Cupertino, CA (US)

(21) Appl. No.: 15/863,866

(22) Filed: Jan. 6, 2018

(51) Int. Cl. H04L 29/08; H04L 29/06; G08B 23/00; A61C 17/22; A61B 5/024; A61B 5/00

(52) U.S. Cl. CPC H04L 67/12; H04L 67/42; G08B 23/00; A46B 9/04; A61B 5/02438; A61B 5/0022; A61C 17/221

(57) ABSTRACT

The IoT Toothbrush will be the first device to interact with the human fingers daily, collecting vital signs using a certain special sensor. The proposed IoT Toothbrush will collect vital signs from the incorporated sensor, which will transfer to the network device's so-called Toothbrush-Hub. The Toothbrush-Hub will utilize Bluetooth technology to communicate with the IoT Toothbrush and Wi-Fi protocol to communicate with the Server Software to monitor the user's health.

### SUMMARY

The IoT Toothbrush is a device used to monitor a user's vital signs through its interaction the user's finger touch with the toothbrush's sensor. The IoT Toothbrush is connected through a Wi-Fi protocol via its associated base, the Toothbrush-Hub in order to send personal identification and vital signs to the individual's personal mobile device. Monitoring the user's vital signs can be tracked via the Client Application downloaded through the user's personal mobile device.

### DESCRIPTION

### Technical Field

The present disclosure relates generally to IoT communications networking to facilitate the process of monitoring human health.

### Background

When the human fingers touch the sensor on the IoT Toothbrush, personal identification and human vital signs are collected and transferred to a simple Bluetooth-Wi-Fi interface device, called the Toothbrush-Hub. This device in turn transfers the collected data to the Server Software through the Wi-Fi interface. The Server Software listens to the Toothbrush-Hub to accept data from this device. The software will save data from each person based on personal identification of the individual user. Overtime, the software will analyze the collected data and archive such historic data. It will alert the individual if their data triggers any health-related risk factors set pursuant to certain pre-defined thresholds (i.e. chronic dehydration from elevated heart rate). Furthermore, abrupt changes in vital signs will also alert the user and any other persons they wish to share such information with (i.e. significant decrease in oxygen saturation in an elderly person.

### BRIEF DESCRIPTION OF FIGURES

For a more complete understanding of the present disclosure and its features and advantages, reference is now made to the following description, taken in conjunction with the accompanying drawing, in which:

FIG. 1 depicts an example of two toothbrushes with two of the associated Toothbrush-Hubs to communicate with the Server Software. The Server Software further interacts with connected mobile devices to provide users with shared historic data.

FIG. 2 depicts the proposed design of the toothbrush and its sensor location. The toothbrush is co-located with its base station, the Toothbrush-Hub.

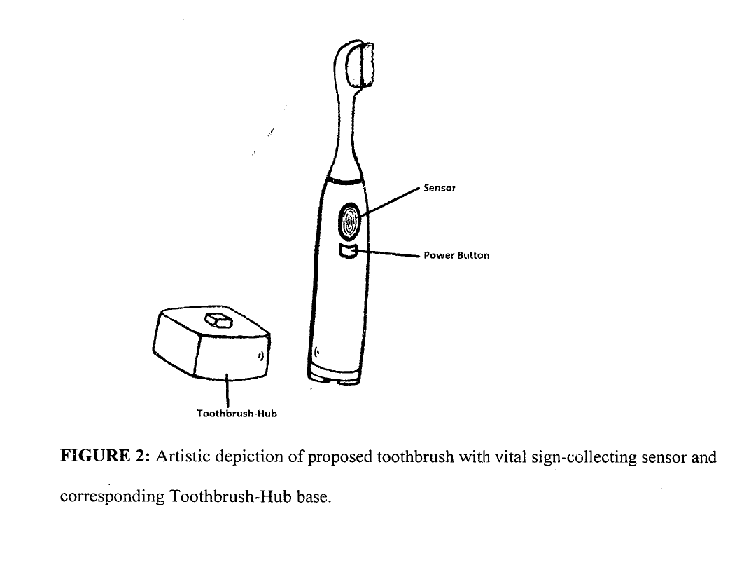

### DESCRIPTION OF EXAMPLE EMBODIMENTS

According to the embodiment, information about a user's vital signs may be collected, analyzed, and disseminated in a comprehensible manner to the user in a simple routine activity that most individuals perform daily. Daily tooth brushing could trigger a complete sequence of events to achieve this task.

Certain embodiments of the disclosure may provide one or more technical advantages. A technical advantage of one embodiment may include the alarm message that is sent to a user periodically until they acknowledge it. Another includes the disclosure of data collected from the IoT Toothbrush to another trusted individual (i.e. monitoring the health of a loved one). Certain embodiments of the disclosure may include none, some, or all of the above technical advantages. One or more other technical advantages may be readily apparent to one skilled in the art from the figures, descriptions, and claims included herein.

### DESCRIPTION

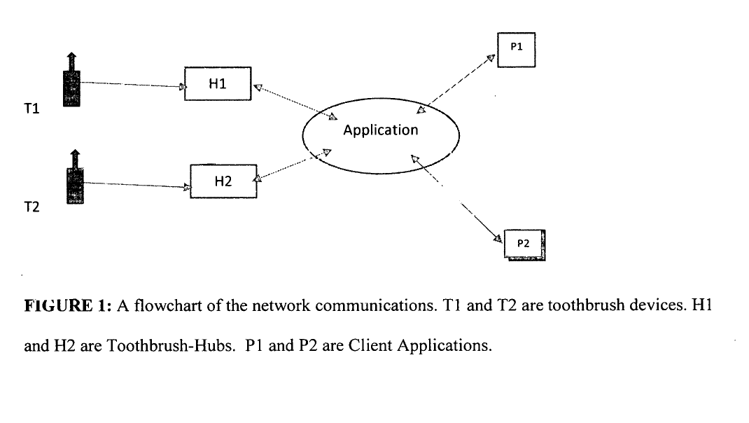

FIG. 1 depicts an example toothbrush T1 and toothbrush T2 equipped with detection sensors. The sensor's location lies at the top of the base of the toothbrush. The human fingers need to touch the sensor during the teeth cleaning. Each toothbrush has a built-in Bluetooth interface which communicates with its corresponding Toothbrush-Hub to exchange the data collected from the sensor. Sensor and Bluetooth functions are activated when the toothbrush is turned on.

Each toothbrush has its associated base station—the Toothbrush-Hub—which in FIG. 1, H1 is the base station of toothbrush T1, and H2 is the base station of toothbrush T2. The Toothbrush-Hubs, H1 and H2, have Bluetooth interface with the IoT Toothbrush and Wi-Fi interface with the Server Software. In general, the Toothbrush-Hub is connected to a power source and is ready to communicate to both IoT toothbrushes and the Server Software. The Toothbrush-Hub acts as a hub between the IoT Toothbrush and Server Software. When the IoT Toothbrush is turned on and the sensor is touched, the Bluetooth session commences and establishes a connection to its Toothbrush-Hub. When the connection between the IoT Toothbrush and Toothbrush-Hub is established, the IoT Toothbrush identifies its user via personal identification and sends the collected vital signs to the Toothbrush-Hub.

The Toothbrush-Hub may maintain a permanent connection with the Server Software using a Wi-Fi protocol. The connection is implementation choice, either a simple User Datagram Protocol/Transmission Control Protocol (“UDP/TCP”) socket or Secure Shell (“SSH”) connection. When the Toothbrush-Hub receives data from the IoT Toothbrush, it packs and sends the packet data into a message that the Server Software can interpret and understand. The Server Software is a software that runs as a server, and its clients are the Toothbrush-Hub and personal mobile devices. The Toothbrush-Hubs, H1 and H2, are registered as source clients. The source clients supply raw data collected from the IoT Toothbrush when the sensor is touched by a human person. The personal mobile devices, P1 and P2, are registered as destination clients. The destination clients receive notifications after raw data gets analyzed. H1, H2, P1, and P2 can use the Dynamic Host Configuration Protocol (“DHCP”) to register with the Server Software. Users will need to install a simple Client Application on their mobile devices to register the service with the Server Software. The Client Application has two phases of registration. The first phase uses the DHCP to establish a connection with the Server Software. The second phase of registration occurs when the user touches the IoT Toothbrush sensor. The Client Application receives personal identification from the Server Software and prompts for confirmation from the individual who touches the sensor. If the person confirms the query upon reviewing its Terms and Conditions and Privacy Policy, the Client Application sends confirmation back to the Server Software. This completes the registration.

To correlate the personal mobile device and IoT Toothbrush personal identification, two phases of registration are needed. The Client Application uses the DHCP protocol to register the personal mobile device's IP address with the Server Software. A person may need to touch the IoT Toothbrush sensor during the second phase destination of client registration. When personal identification is sent to the Server Software, it broadcasts to all registered mobile devices the personal identification. The Server Software then waits for confirmation from the Client Application for confirmation to the Server Software registers the personal identification with the personal mobile device.

At any given time, when the person uses the toothbrush, raw data collected from the sensor is sent to the Server Software via the Toothbrush-Hub. The Server Software analyzes the raw data for any abnormal behavior, which will prompt the Server Software to send notifications to the mobile device, alarming the person to take appropriate action.

What is claimed is:

1. (1) location, by the toothbrush, a sensor is attached to the top of the base of the toothbrush to collect personal data and vital signs
(2) location, via the base station near the toothbrush to collect and transfer raw personal data and vital signs to the central Server Software
(3) register, two-phase toothbrush registration, a method to correlate toothbrush and personal mobile device(s)
Modifications, additions, or omissions may be made to the systems, apparatuses, and methods disclosed herein without departing from the scope of the invention. The components of the systems may be integrated or separated. Moreover, the operations of the systems may be performed by more, fewer, or other components. Additionally, operations of the systems may be performed using any suitable logic comprising software, hardware, and/or other logic. The methods may include more, fewer, or other steps. Additionally, steps may be performed in any suitable order.
Although this disclosure has been described in terms of certain embodiments, alterations and permutations of the embodiments will be apparent to those skilled in the art. Accordingly, the above description of the embodiments does not constrain this disclosure. Other changes, substitutions, and alterations are possible without departing from the spirit and scope of this disclosure, as defined by the following claims.
